(19) United States
(12) Patent Application Publication
(10) Pub. No.: US 2015/0025590 A1
(43) Pub. Date: Jan. 22, 2015

(54) PHYSICAL HEALTH CAP APPLICATION SYSTEM

(71) Applicant: Chih-Lung Cheng, Tainan City (TW)

(72) Inventor: Chih-Lung Cheng, Tainan City (TW)

(21) Appl. No.: 13/943,807

(22) Filed: Jul. 17, 2013

(51) Int. Cl. A61N 1/36

(52) U.S. Cl. CPC A61N 1/36021
USPC 607/3; 607/139

(57) ABSTRACT

A physical health cap application system include an electrode cap configured to fit the head of any person, and a main unit including a DC-stimulator plus and a positive and negative DC current generator unit electrically connected to the electrode cap for controlling the electrode cap to discharge a direct electrical current into predetermined regions of interest in the cerebral cortex of the head of a person to help relieve pain.

### BACKGROUND OF THE INVENTION

(1) Field of the Invention

The present invention relates to physical health care technology, and more particularly to a physical health cap application system for brain health care, which comprises an electrode cap, and a main unit electrically connected to the electrode cap for controlling the electrode cap to discharge a direct electrical current into predetermined regions of interest in the cerebral cortex of the head of a person to help relieve pain.

(2) Description of the Prior Art

The brain is the center of the nervous system that controls all bodily functions. Brain sciences and related programs have been widely discussed. In addition to the known basic brain structures and their functions and development, studies on human brain-cognitive abilities are conducted continuously. Under the assistance of various physical and mental health measurement tools, researches in this field become more diverse, objective and complete. Many research reported that the electroencephalogram (EEG) is a reliable test to assess brain activity. It is used to show the type and location of the activity in the brain during a seizure. It also is used to evaluate people who are having problems associated with brain function. EEG is also used in the diagnosis of coma, encephalopathies, and brain death. Civilization stress and bad living habits can cause body aches. In addition to medication, acupuncture and physical therapy can help relieve pain.

Transcranial Direct-Current Stimulation (tDCS) is a technique that involves passing low-level electrical currents through parts of the brain. tDCS was originally developed to help patients with brain injuries such as strokes. Tests on healthy adults demonstrated that tDCS can increase cognitive performance on a variety of tasks. tDCS has been utilized to enhance language and mathematical ability, attention span, problem solving, memory, and coordination.

### SUMMARY OF THE INVENTION

The present invention has been accomplished under the circumstances in view. It is therefore the main object of the present invention to provide a physical health cap application system, which is practical for stimulating predetermined regions of interest in the cerebral cortex of the head of a person to help relieve pain.

To achieve this and other objects of the present invention, a physical health cap application system of the invention comprises an electrode cap, and a main unit electrically coupled with the electrode cap. The main unit comprises a DC-stimulator plus and a positive and negative DC current generator unit. The electrode cap comprises a plurality of mounting portions, and a plurality of electrode units respectively mounted in the mounting portions and electrically connected to the main unit. The main unit controls the electrode cap to discharge a direct electrical current into predetermined regions of interest in the cerebral cortex of the head of a person to help relieve pain.

Further, each electrode unit comprises a probe holder block defining therein a recess, a plurality of metal tension springs mounted in the recess of the probe holder block and arranged in an array, a plurality of probes respectively supported on the metal tension springs and respectively electrically connected to said main unit. Subject to the effects of the torsion springs, the probes can be positively and comfortably attached to the skin of the user's head to stimulate the selected regions of interest.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an exploded view of an electrode cap for physical health cap application system in accordance with the present invention.

FIG. 2 is an exploded view of an electrode unit for physical health cap application system in accordance with the present invention.

FIG. 3 is a schematic drawing illustrating an application status of a physical health cap application system in accordance with the present invention.

FIG. 4 is a schematic drawing of the present invention, illustrating an arrangement of electrodes in the electrode cap.

FIG. 5 is a schematic drawing of the present invention, illustrating another arrangement of electrodes in the electrode cap.

FIG. 6 illustrates the arrangement of regions of interest corresponding to the cerebral cortex.

### DETAILED DESCRIPTION OF THE PREFERRED EMBODIMENTS

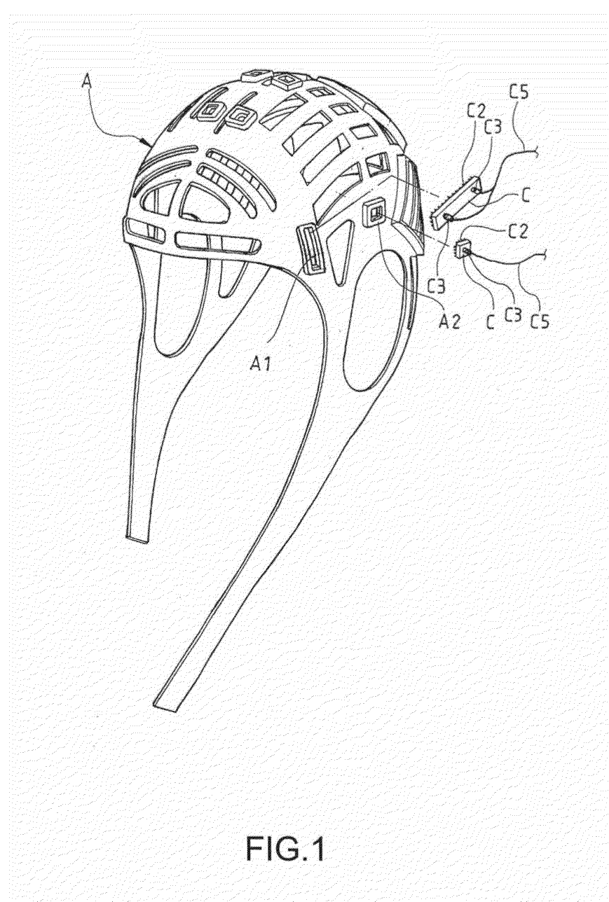

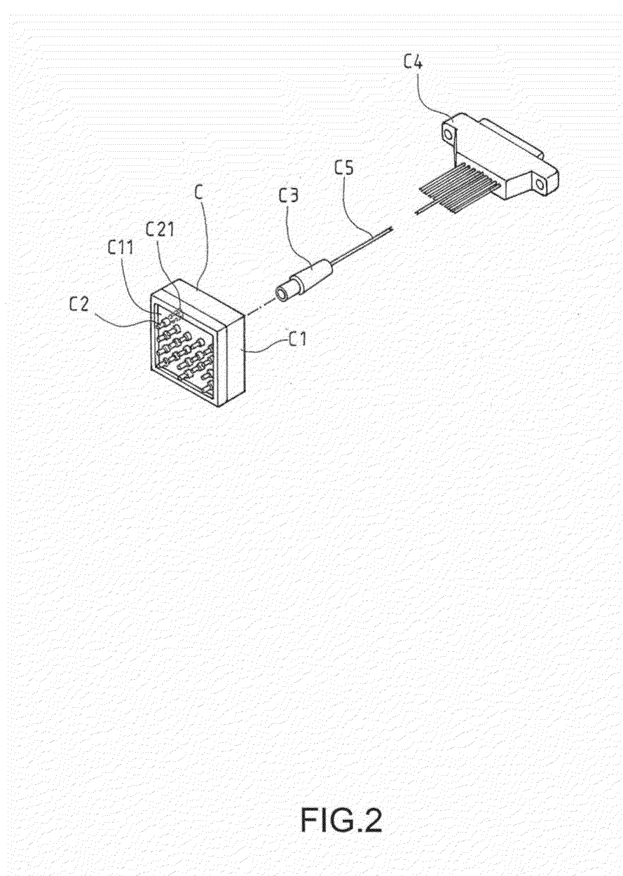

Referring to FIGS. 1, 2 and 3, a physical health cap application system in accordance with the present invention is shown. The physical health cap application system comprises an electrode cap A, and a main unit B electrically coupled with the electrode cap A. The electrode cap A is a rubber cap configured to fit the head of a person, comprising a plurality of mounting portions A1/A2 of, for example, rectangular shape, respectively disposed corresponding to regions of interest in the left and right parietal lobes, and a plurality of electrode units C respectively mounted in the mounting portions A1/A2 and electrically connected to the main unit B. Each electrode unit C comprises a probe holder block C1 defining a recess C11, a plurality of metal tension springs C21 mounted in the recess C11 of the probe holder block C1 and arranged in an array, a plurality of probes C2 respectively supported on the metal tension springs C21 and insertable through open spaces in the hair of the user's head into contact with the skin of the user's head around regions of interest in the left and right parietal lobes, an electrical connector C4 electrically connected to the main unit B, and a plurality of metal conducting terminals C3 and lead wires C5 respectively electrically connected in series between the metal tension springs C21 and the electrical connector C4. Because the probes C2 are respectively supported on the metal tension springs C21, the electrode cap A renders a high level of wearing comfort. The main unit B comprises a DC-stimulator plus and a positive and negative DC current generator unit. The invention can also be configured to simulate the regions of interest in the left and right parietal lobes by: vibration, warm heat, electricity, laser, acoustic waves, electrical waves, and/or press.

Subject to the operation of the DC-stimulator plus of the main unit B, the electrode cap A discharges controlled electricity to regions of interest in the left and right parietal lobes of the user's head to stimulate the regions of interest in the left and right parietal lobes, enhancing brain functions and helping relieve pains.

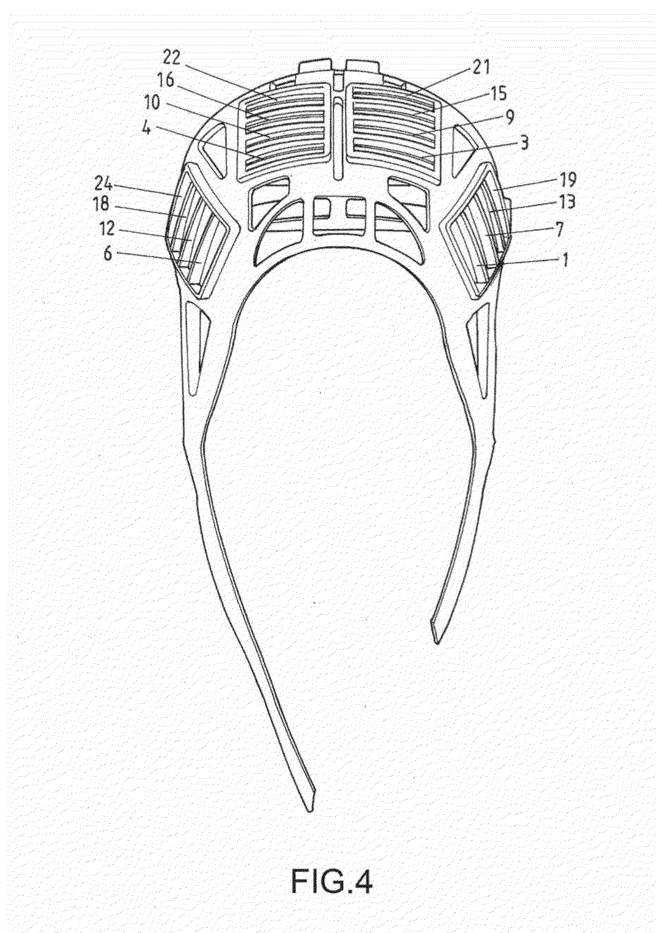

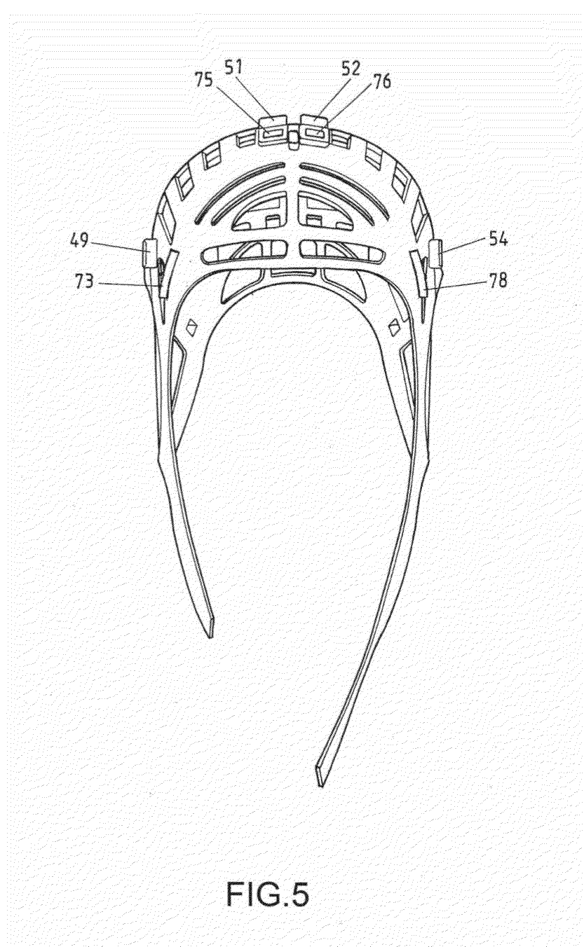

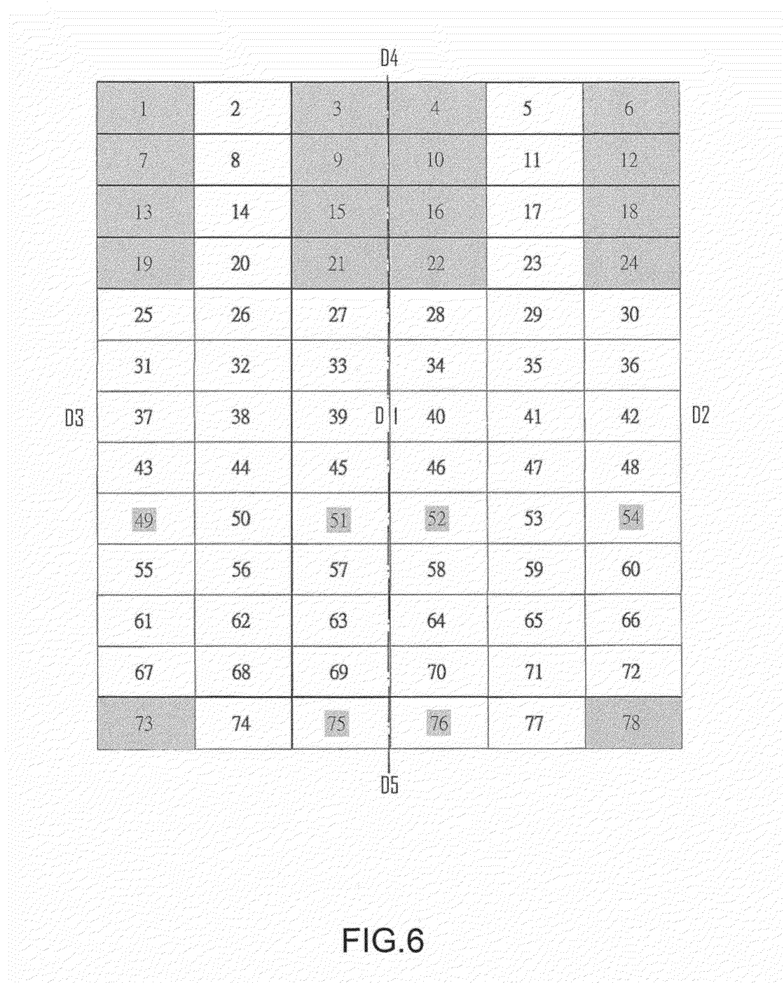

Referring to FIGS. 4, 5 and 6, the head of a human being is divided into the parietal area D1 on the top side, the right temporal lobe area D3 on the right side, the left temporal lobe area D2 on the left side, the occipital area D4 on the back side, and the frontal area D5 on the front side. The health care operating area of the present invention is located at the parietal scalp of the skullcap. The front-back parietal dimension is equally divided into 13 horizontal regions. The left-right parietal dimension is equally divided into 6 horizontal regions. Thus, the combination of the two dimensions provides total 78 regions of interest in the parietal that are numbered from 1 through 78 as follows:

Right parietal area: 1, 2, 3, 7, 8, 9, 13, 14, 15, 19, 20, 21, 25, 26, 27, 31, 32, 33, 37, 38, 39, 43, 44, 45, 49, 50, 51, 55, 56, 57, 61, 62, 63, 67, 68, 69, 73, 74, 75.

Left parietal area: 4, 5, 6, 10, 11, 12, 16, 17, 18, 22, 23, 24, 28, 29, 30, 34, 35, 36, 40, 41, 42, 46, 47, 48, 52, 53, 54, 58, 59, 60, 64, 65, 66, 70, 71, 72, 76, 77, 78.

The parietal area D1 is divided into total 78 regions of interest that are covered within the health care operating area of the present invention. The invention is adapted to give different stimulation to these 78 regions of interest, achieving optimal brain health care effects.

Due to complicated ling relationship between the bones and the muscles in the body and the stretching variations of muscle dynamics and contralateral relationship, there is considerable variation in health care. In the variation, there is still a basic trajectory to follow. The locations of pain and illness can be allocated subject to nervous reflex. Therefore, it is necessary to apply different stimulations on different positions of the aforesaid 78 regions of interest for relieving pains and treating illness. Some regions of interest are defined as main stimulation regions while the other regions of interest are defined as supplementary stimulation regions. If stimulation on the main stimulation regions cannot achieve a significant result, stimulation on the supplementary stimulation regions can then be applied.

The electrode cap A selects regions 1, 3, 4, 6, 7, 9, 10, 12, 13, 15, 16, 18, 19, 21, 22, 24, 73 and 78 to match with 1×4 cm electrode units C and regions 49, 51, 52, 54, 75 and 76 to match with 1×1 cm electrode units C for working. By means of 24 electrode loops in the electrode cap A to generate electrical stimulation, the invention effectively stimulate the functions of a specific region of the cerebral cortex of the parietal area to help relieve pain and treat illness.

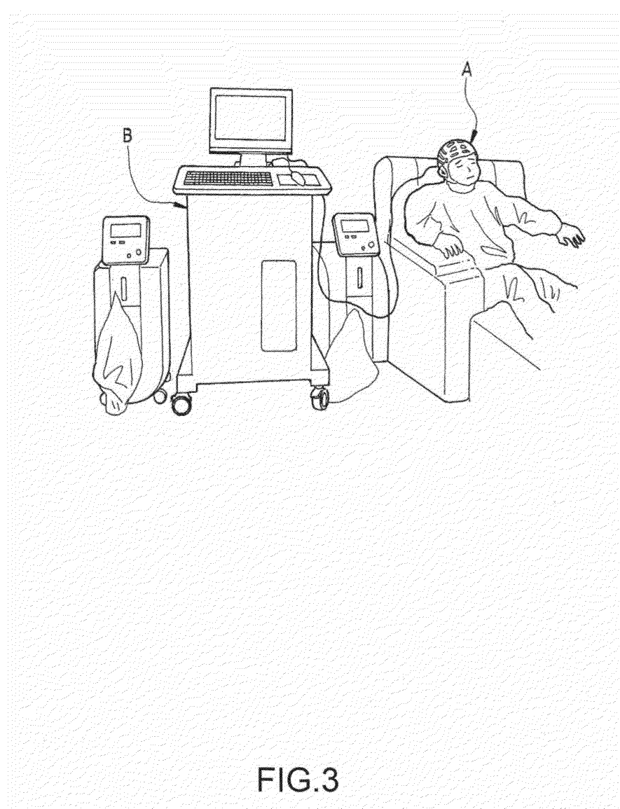

Referring to FIG. 3, when using the physical health cap application system, put the electrode cap A on the user's head, and then electrically connect the electrode cap A to the main unit B. The system is formed of two parts. One is the DC-stimulator plus. The other is positive and negative DC loop. The DC-stimulator plus generate electricity to the 24 electrode loops in the electrode cap A to stimulate the selected specific region of the cerebral cortex of the parietal area, achieving pain relieving and illness treating effects.

The application of transcranial direct-current stimulation (tDCS) is a non-invasive technique widely used in medical centers for medical research studies. This technique is commonly used in cortical plasticity research studies to stimulate regions of interest of the cerebral cortex of the parietal are by means of inputting a direct electric current through the probes C2. Further, the electrode cap A is made out of elastic rubber. It can be conveniently adjusted to fit different users. After put on the user's head, it can be positively kept in position.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A physical health cap application system, comprising an electrode cap and a main unit electrically coupled with said electrode cap, said main unit comprising a DC-stimulator plus and a positive and negative DC current generator unit, said electrode cap comprising a plurality of mounting portions and a plurality of electrode units respectively mounted in the mounting portions and electrically connected to said main unit, each said electrode unit comprising a plurality of spring-loaded probes controlled by said main unit to discharge a direct electrical current to simulate predetermined regions of interest in the cerebral cortex of the head of a person;
wherein the physical health cap application system divides the head of a person into a parietal area on a top side, a right temporal lobe area on a right side, a left temporal lobe area on a left side, an occipital area on a back side and a frontal area on a front side; the front-back parietal dimension is equally divided into 13 horizontal regions; the left-right parietal dimension is equally divided into 6 horizontal regions; the combination of said front-back and left-right parietal dimensions provides total 78 regions of interest in the parietal that are numbered from 1 through 78; said electrode cap selects the regions of interest of 1, 3, 4, 6, 7, 9, 10, 12, 13, 15, 16, 18, 19, 21, 22, 24, 73 and 78 to match with said electrode units for working and the regions of interest of 49, 51, 52, 54, 75 and 76 to match with said electrode units for working; said electrode cap uses 24 electrode loops therein for generating electrical stimulation to stimulate the functions of predetermined regions of the cerebral cortex of the parietal area.

2. The physical health cap application system as claimed in claim 1, wherein said electrode cap is made out of elastic rubber and configured to fit different users.

3. The physical health cap application system as claimed in claim 1, wherein each said electrode unit comprises a probe holder block, a plurality of metal tension springs mounted in said probe holder block and arranged in an array, a plurality of probes respectively supported on said metal tension springs and respectively electrically connected to said main unit.

4. The physical health cap application system as claimed in claim 1, wherein the probe holder block of each said electrode unit defines therein a recess configured to hold said metal tension springs and said probes.

5. The physical health cap application system as claimed in claim 1, wherein said electrode cap is configured to simulate the predetermined regions of the cerebral cortex of the parietal area by: vibration, warm heat, electricity, laser, acoustic waves, electrical waves, and/or press.

6. The physical health cap application system as claimed in claim 1, wherein said electrode cap selects the regions of interest of 3, 4, 9, 10, 15, 16, 21, 22, 73 and 78 as main stimulation regions, and the regions of interest of 1, 7, 13, 19, 6, 12, 18 and 24 as supplementary stimulation regions.

7. The physical health cap application system as claimed in claim 1, wherein said electrode cap selects the regions of interest of 3, 4, 15, 16, 73 and 78 as main stimulation regions, and the regions of interest of 1, 6, 13, and 18 as supplementary stimulation regions.

8. The physical health cap application system as claimed in claim 1, wherein said electrode cap selects the regions of interest of 3, 4, 15, 16, 73 and 78 as main stimulation regions, and the regions of interest of 1, 6, 13, 18, 49, 51, 52 and 54 as supplementary stimulation regions.

9. The physical health cap application system as claimed in claim 1, wherein said electrode cap selects the regions of interest of 3, 4, 21 and 22 as main stimulation regions, and the regions of interest of 1, 6, 19, 24, 73 and 78 as supplementary stimulation regions.
